(19) United States
(12) Patent Application Publication
(10) Pub. No.: US 2022/0015712 A1
(43) Pub. Date: Jan. 20, 2022

(54) Biosignal Monitoring System With Motion Artifact Reduction

(71) Applicants: IMEC VZW, Leuven (BE); Stichting IMEC Nederland, Eindhoven (NL)

(72) Inventors: Roland Van Wegberg, Oss (NL); Nick Van Helleputte, Korbeek-Dijle (BE)

(21) Appl. No.: 17/376,543

(22) Filed: Jul. 15, 2021

(30) Foreign Application Priority Data
Jul. 16, 2020 (EP) 20186267.9

(51) Int. Cl. A61B 5/00; A61B 5/308; A61B 5/31; A61B 5/313

(52) U.S. Cl. CPC A61B 5/7207; A61B 5/7225; A61B 5/313; A61B 5/31; A61B 5/308

(57) ABSTRACT

The disclosure includes a biosignal monitoring system for reducing a motion artifact from a biopotential electrical signal input, including a signal processing module, a motion artifact extraction module, and a subtraction module. The motion artifact extraction module and the signal processing module receive the biopotential electrical signal input and the subtraction module receives an extracted signal from an output of the motion artifact extraction module and a biopotential electrical signal from an output of the signal processing module. The subtraction module subtracts the extracted signal from the biopotential electrical signal. The motion artifact extraction module is an analog domain electronic circuit and includes a filter network configured for attenuating differential mode signals of the biopotential electrical signal input from a first frequency, and passing the motion artifact signal from the biopotential electrical signal input up to a second frequency at the output of the motion artifact extraction module.

### CROSS-REFERENCE TO RELATED APPLICATIONS

The present application is a non-provisional patent application claiming priority to European Patent No. 20186267.9, filed on Jul. 16, 2020, the contents of which are hereby incorporated by reference.

### FIELD OF THE DISCLOSURE

The present disclosure is generally related to electronic systems for monitoring body signals and more specifically to biosignal monitoring systems with motion artifact reduction.

### BACKGROUND

Monitoring of biopotential electrical signals, such as electrocardiogram (ECG), electroencephalogram (EEG) or electromyogram (EMG) signals, may be used, for example, to evaluate the body condition of a person. When the biopotential electrical signals are monitored using electrodes attached to the body, such as wet (gel) electrodes or dry electrodes, one of the major motion artifact signals is caused by the change in electrical properties of the skin-electrode/electrolyte interface that are known to have high correlation with motion/movement artifacts, for example by running or stretching the skin.

In the document “Real Time Digitally Assisted Analog Motion Artifact Reduction in Ambulatory ECG Monitoring System”, by Sunyoung Kim et al., 34th Annual International Conference of the IEEE EMBS, San Diego, Calif. USA, 28 Aug.-1 Sep., 2012, a known technique for motion artifact removal is explained by first measuring electrode-skin impedance to adopt it as a reference signal for the digital adaptive filter and then generate an estimated motion artifact signal which is subtracted from the measured biopotential signal in a programmable gain amplifier.

Another known technique for motion artifact removal by reducing the noise reference signal from an accelerometer in an adaptive filter is explained in the document “Using an Adaptive Filter to Remove ECG Motion Artifact Interference”, by Li-Ming Bai et al., 2018 IEEE International Conference on Consumer Electronics-Taiwan (ICCE-TW).

### SUMMARY

The present disclosure provides a biosignal monitoring system for reducing a motion artifact from at least one biopotential electrical signal input, the biosignal monitoring system comprising a signal processing module, a motion artifact extraction module and a subtraction module, wherein the motion artifact extraction module and the signal processing module receive the biopotential electrical signal input and the subtraction module receives both an extracted signal from an output of the motion artifact extraction module and a biopotential electrical signal from an output of the signal processing module; and the subtraction module is configured for subtracting the extracted signal from the biopotential electrical signal; wherein the motion artifact extraction module is implemented as analog domain electronic circuit and comprises a filter network, configured for attenuating differential mode signals of the biopotential electrical signal input from a first frequency, and passing a representation of the motion artifact signal from the biopotential electrical signal input up to a second frequency at the output of the motion artifact extraction module. According to an example, the system allows for extraction of the motion artifact signal more accurately and/or power efficiently. According to an example, the system is suitable for single-end motion artifact reduction.

According to an example embodiment, the filter network comprises a first forward path amplifier having a first input connected to a first biopotential electrical signal input terminal, a second input and an output connected to the first output terminal of the motion artifact extraction module; and a second forward path amplifier having a first input connected to a second biopotential electrical signal input terminal, a second input and an output connected to the second output terminal of the motion artifact extraction module. The filter network further comprises a capacitor between the outputs of the first and second forward path amplifiers. The filter network has a characteristic of a low pass filter for a differential mode signal and still passes a representation of a motion artifact signal.

According to an example embodiment, the capacitor in the filter network has a value between 100 nF and 100 μF. According to an example embodiment, the transconductance of the forward path amplifiers in the filter network has a value between 10 nS and 1000 nS. According to an example embodiment, the forward path amplifiers are implemented as transconductance amplifiers or gₘ-stages.

According to an example embodiment, the motion artifact extraction module, the signal processing module, and the subtraction module are implemented as analog domain electronic modules. According to still another example embodiment, the signal processing module and the subtraction module are implemented as an instrumentation amplifier; the biopotential electrical signal input is connected to both a first input of the instrumentation amplifier and an input of the motion artifact extraction module; the output of the motion artifact extraction module is connected to a second input of the instrumentation amplifier.

According to an example embodiment, the subtraction module is implemented as a digital domain electronic module. According to an example, the system is further able to reduce the motion artifact signal in a more controlled way in the digital domain with adapted gains for different frequency.

According to still another example embodiment, the signal processing module is implemented as an instrumentation amplifier, the biosignal monitoring system further comprises: a first analog-to-digital converter connected to the output of the motion artifact extraction module and further connected to a first input of the subtraction module; and a second analog-to-digital connected to the output of the signal processing module and further connected to a second input of the subtraction module.

According to still another example embodiment, the motion artifact extraction module is integrated in an instrumentation amplifier further comprising a third forward path amplifier connected between one input terminal of the motion artifact extraction module and the biopotential electrical signal input terminal; and a fourth forward path amplifier connected between the other input terminal of the motion artifact extraction module and the biopotential electrical signal input terminal; a first analog-to-digital converter connected to the output of the motion artifact extraction module and further connected to a first input of the subtraction module; and a second analog-to-digital converter connected to the output of the signal processing module and further connected to a second input of the subtraction module.

According to an example embodiment, the subtraction module is implemented using a least mean squares algorithm.

According to an example embodiment, the biopotential electrical signal input is an ECG, EEG, or EMG signal input.

According to an example embodiment, a microchip comprises the biosignal monitoring system. According to an example embodiment, a biomedical device comprises the microchip or the biosignal monitoring system.

According to an example embodiment, a method for reducing a motion artifact from at least one biopotential electrical signal input in the biosignal monitoring system comprises the steps of extracting a representation of the motion artifact signal from the biopotential electrical signal input by attenuating a differential mode signal present in the biopotential electrical signal input from a first frequency and passing a representation of the motion artifact signal from the biopotential electrical signal input up to a second frequency; and subtracting the extracted representation of the motion artifact signal from the biopotential electrical signal input.

These as well other aspects, advantages, and alternatives will become apparent to those of ordinary skill in the art by reading the following detailed description, with reference where appropriate to the accompanying drawings.

### BRIEF DESCRIPTION OF THE FIGURES

The above, as well as additional, features will be better understood through the following illustrative and non-limiting detailed description of example embodiments, with reference to the appended drawings.

The disclosure will be further elucidated by means of the following description and the appended figures. Various exemplary embodiments are described herein with reference to the following figures, wherein like numeral denotes like entities. The figures described are schematic and are non-limiting. Further, any reference signs in the claims shall not be construed as limiting the scope of the present disclosure. Still further, in the different figures, the same reference signs refer to the same or analogous elements.

FIG. 1 is a schematic diagram of a biosignal monitoring system for reducing motion artifacts from at least one biopotential electrical signal input, according to an example.

FIG. 2a is a schematic diagram of a motion artifact extraction module, according to an example.

FIG. 2b is a schematic diagram of a filter network circuit in the motion artifact extraction module presented in FIG. 2a, according to an example.

FIG. 2c shows an example of the gain at output terminals corresponding to different frequencies in respect to the differential mode signal or the single-end motion artifact signal from biopotential electrical signal input terminals, according to an example.

FIG. 3 is a schematic diagram of a biosignal monitoring system for reducing motion artifacts from at least one biopotential electrical signal input, according to an example.

FIG. 4 is a schematic diagram of a biosignal monitoring system for reducing motion artifacts from at least one biopotential electrical signal input, according to an example.

FIG. 5 is a schematic diagram of a biosignal monitoring system for reducing motion artifacts from at least one biopotential electrical signal input, according to an example.

All the figures are schematic, not necessarily to scale, and generally only show parts which are necessary to elucidate example embodiments, wherein other parts may be omitted or merely suggested.

### DETAILED DESCRIPTION

Example embodiments will now be described more fully hereinafter with reference to the accompanying drawings. That which is encompassed by the claims may, however, be embodied in many different forms and should not be construed as limited to the embodiments set forth herein; rather, these embodiments are provided by way of example. Furthermore, like numbers refer to the same or similar elements or components throughout.

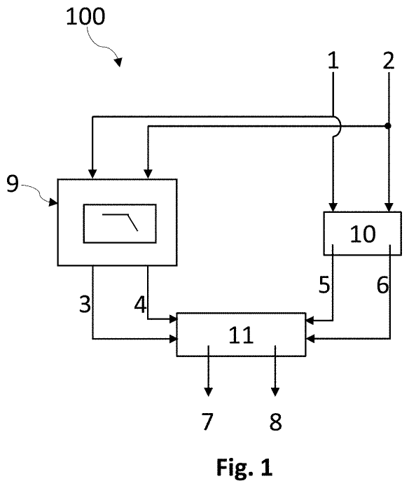

FIG. 1 is a schematic diagram of a biosignal monitoring system 100 for reducing a motion artifact from at least one biopotential electrical signal input 1, 2, according to an example. The biosignal monitoring system 100 comprises three modules: a signal processing module 10, a motion artifact extraction module 9 including a filter network, and a subtraction module 11.

The signal processing module 10 and the motion artifact extraction module 9 are configured for receiving the biopotential electrical signal input 1, 2 from corresponding electrodes, of a biomedical recording device attached to a body. The biopotential electrical signal input 1, 2 comprises a differential mode signal and may also comprise a motion artifact signal. The motion artifact signal can be a single-end motion artifact signal which is the motion artifact signal from one terminal of the biopotential electrical signal input 1, 2 with respect to the ground. The output 5, 6 of the signal processing module 10 comprises at least a differential mode signal and may comprise a motion artifact signal.

The motion artifact extraction module 9 is implemented as an analog domain electronic circuit and comprises a filter network which is configured for attenuating differential mode signals of the biopotential electrical signal input 1, 2 from a first frequency F1, and passing a signal gain difference up to a second frequency F2 at the output 3, 4 of the motion artifact extraction module 9, which is a representation of the motion artifact signal from the biopotential electrical signal input 1, 2. Thus, the motion artifact signal is extracted from the biopotential electrical signal input 1, 2 and provided at the output 3, 4 of the motion artifact extraction module 9. Thus, the output 3, 4 of the motion artifact extraction module 9 comprises at least a representation of the motion artifact signal and an attenuated differential mode signal.

The output 3, 4 of the motion artifact extraction module 9 is connected to a first input of the subtraction module 11 and the output 5, 6 of the signal processing module 10 is connected to a second input of the subtraction module 11. The subtraction module 11 is configured for subtracting the signals at the output 3, 4 of the motion artifact extraction module 9 from the signals at the output 5, 6 of the signal processing module 10. The output 7, 8 of the subtraction module comprises at least a biopotential electrical signal with the motion artifact signal being reduced.

According to an example embodiment, the signal processing module 10 may adapt, process, filter, and/or further amplify the biopotential electrical signal input 1, 2.

According to an example embodiment, the system 100 may be in a biomedical recording device, for example: an ECG, EEG, or EMG recording device. Thus, the biopotential electrical signal input 1, 2 may be an ECG, EEG, or EMG signal input.

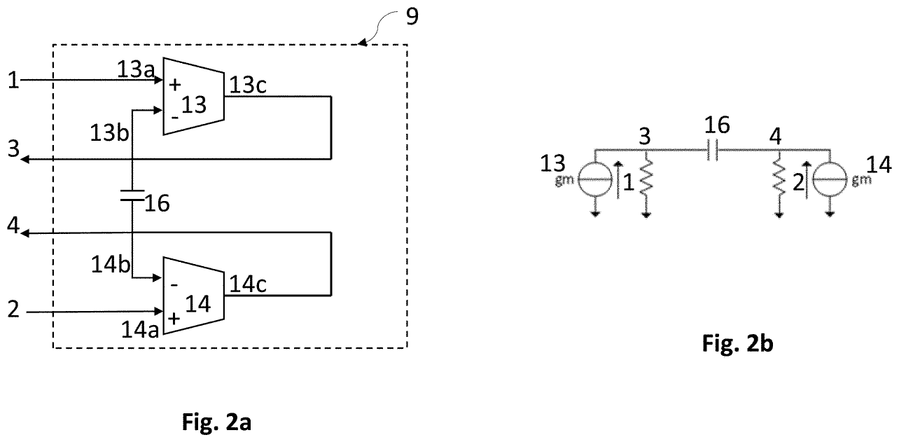

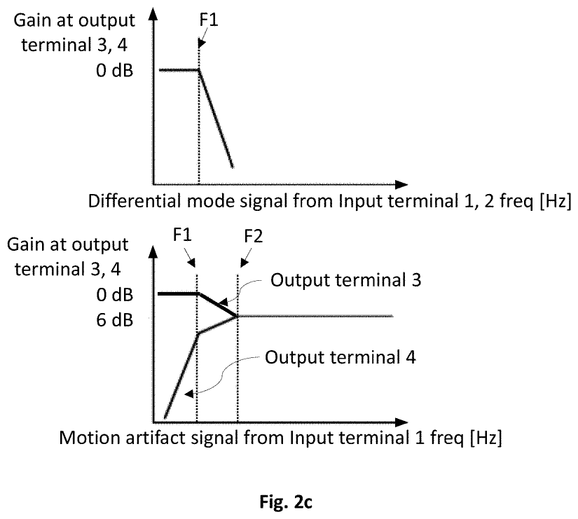

FIG. 2a is a schematic diagram of the motion artifact extraction module 9 including a filter network, according to an example. The filter network comprises a first forward path amplifier 13, a second forward path amplifier 14, and a capacitor 16 between the outputs 13c, 14c of the forward path amplifiers 13, 14.

According to an example embodiment, the forward path amplifiers 13, 14 each have a non-inverting input and an inverting input. The biopotential electrical signal input 1, 2 is connected to the non-inverting inputs 13a, 14a. The output 13c of the first forward path amplifier 13 is connected to the inverting input 13b and the output terminal 3 of the motion artifact extraction module 9; the output 14c of the second forward path amplifier 14 is connected to the inverting input 14b and the output terminal 4 of the motion artifact extraction module 9. The capacitor 16 is connected between outputs 13c, 14c, which is also the inverting inputs 13b, 14b, of the forward path amplifiers 13, 14. Thus, the forward path amplifier 13 and the capacitor 16 function as a gₘ-C low pass filter for the signal at the biopotential electrical signal input 1, and the forward path amplifier 14 and the capacitor 16 function as a gₘ-C low pass filter for the signal at the biopotential electrical signal input 2.

FIG. 2b is a schematic diagram of the filter network circuit in the motion artifact extraction module 9 presented in the schematic diagram of FIG. 2a. The output impedances are also shown in the circuit (not shown in the FIG. 2a).

According to an example embodiment, the transconductance is the same for the forward path amplifiers 13 and 14. The output impedance R is assumed to be the same for the output terminals 3 and 4.

The filter network has a low pass filter characteristic with a cut-off frequency at a first frequency F1. The transfer function with respect to the differential mode signal from the biopotential electrical signal input 1, 2 to output 3, 4 is:

$$\frac{V o u {t}_{D M}}{V i {n}_{D M}} = \frac{{g}_{m} * R}{( 1 + {g}_{m} * R ) + s * R * 2 C}$$

$$and$$

$$pole s = \frac{- {g}_{m}}{2 C}$$

In this equation: Vout,DM is the output voltage of the filter network with respect to the differential mode signal at the biopotential electrical signal input 1, 2, Vin,DM is the input voltage of the filter network with respect to the differential mode signal at the biopotential electrical signal input 1, 2, gₘ is the transconductance gain of the forward path amplifiers, C is the capacitance of the capacitor 16, and R is the output impedance of output terminals 3, 4 (shown in the FIG. 2b).

According to an example embodiment, the motion artifact signal is a single-end motion artifact signal at the biopotential electrical signal 1. The filter network has different gain responses at the output terminal 3 and 4 with respect to the motion artifact signal. The transfer function with respect to the motion artifact signal from the biopotential electrical signal 1 to output terminal 3 is (it is applicable to the motion artifact signal from the biopotential electrical signal 2 to output terminal 4):

$$\frac{V o u t {P}_{M A}}{V i n {P}_{M A}} = \frac{{g}_{m} * R * ( 1 + {g}_{m} * R + s * R * C )}{( 1 + {g}_{m}^{2} * {R}^{2} ) + s * {g}_{m} * {R}^{2} * 2 C}$$

$$and$$

$$pole s = \frac{- {g}_{m}}{2 C} , \\ zero s = \frac{- {g}_{m}}{C}$$

In this equation: VoutP_{MA} is the output voltage at output terminal 3 with respect to the motion artifact signal at the biopotential electrical signal input 1, and VinP_{MA} is the input voltage of the motion artifact signal at the biopotential electrical signal input 1.

And the transfer function of the motion artifact signal from biopotential electrical signal 1 to output terminal 4 is (it is applicable to the motion artifact signal from biopotential electrical signal 2 to output terminal 3):

$$\frac{V o u t {N}_{M A}}{V i n {P}_{M A}} = \frac{s * {g}_{m} * {R}^{2} * 2 C}{( 1 + {g}_{m}^{2} * {R}^{2} ) + s * {g}_{m} * {R}^{2} * 2 C}$$

$$and$$

$$pole s = \frac{- {g}_{m}}{2 C} , \\ zero s = 0$$

In this equation: VoutN_{MA} is the output voltage of output 4 with respect to the motion artifact signal at the biopotential electrical signal input 1.

FIG. 2c shows an example of the gain at output terminal 3, 4 corresponding to different frequencies with respect to the differential mode signal or the single-end motion artifact signal of the biopotential electrical signal input terminal 1, 2.

As indicated in the transfer functions, the first frequency F1 is equal to gₘ/2C for differential signals and for the motion artifact signal. The signals at output terminal 3 and 4 of the motion artifact extraction module 9 have different gain responses with respect to the motion artifact signal, and the gain difference is the representation of the motion artifact signal. The gain at the output terminal 3 and 4 of the motion artifact extraction module 9 reaches the same value at the second frequency F2, which is equal to gₘ/C, and remains the same at frequencies greater than the second frequency F2. The first frequency F1 and the second frequency F2 therefore generally cannot be designed independently of each other. By properly designing capacitor 16 and the forward path transistors of the feedback network filter, the first frequency F1 and the second frequency F2 can be tuned according to need. The output 3, 4 includes a representation of the motion artifact signals up to the second frequency F2 and an attenuated low-frequency differential signal from the first frequency F1.

The motion artifact signals are in the frequency range of up to 4 Hz with the most artifacts in the frequency range around 2 Hz. According to an example embodiment, if the first frequency F1 is tuned to 0.5 Hz, the second frequency F2 is accordingly equal to 1 Hz, and the output 3, 4 of the motion artifact extraction module 9 comprises the differential mode signal attenuated from 0.5 Hz and a representation of the motion artifact signal up to 1 Hz. According to an example embodiment, if the first frequency F1 is tuned to 2 Hz, the second frequency F2 is accordingly equal to 4 Hz, and the output 3, 4 of the motion artifact extraction module 9 comprises the differential mode signal attenuated from 2 Hz and a representation of the motion artifact signal up to 4 Hz.

According to an example embodiment, the capacitor 16 may have a capacitance between about 100 nF and 100 μF. According to an example embodiment, the transconductance amplifiers have a transconductance between about 10 nS and 1000 nS (nano Siemens).

According to an example embodiment, the forward path amplifiers 13, 14 are transconductance amplifiers or gₘ-stages.

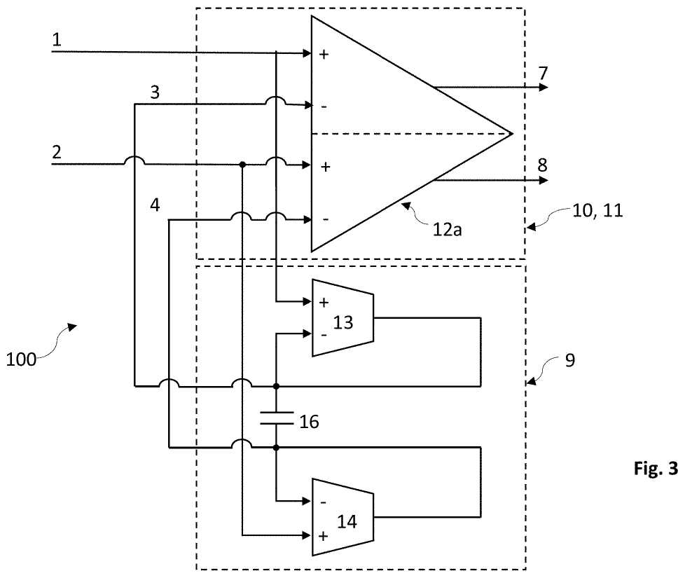

FIG. 3 shows a first schematic diagram of a biosignal monitoring system 100 for reducing a motion artifact from at least one biopotential electrical signal input 1, 2 according to an example embodiment. The system 100 comprises a biopotential electrical signal input 1, 2, an instrumentation amplifier 12a, a signal processing module 10, a motion artifact extraction module 9 including a filter network, and a subtraction module 11.

According to an example embodiment, the motion artifact extraction module 9, the signal processing module 10, and the subtraction module 11 in the system 100 are implemented as analog domain electronic modules.

According to an example embodiment, both the signal processing module 10 and the subtraction module 11 are implemented inside the instrumentation amplifier 12a. Thus, the input of the instrumentation amplifier 12a is the input 1, 2 of the signal processing module 10, the output of the instrumentation amplifier 12a is the output 7, 8 of the subtraction module 11, and the output 5, 6 of the signal processing module 10 becomes an internal output inside the instrumentation amplifier 12a (not shown in FIG. 3). The biopotential electrical signal input 1, 2 is connected to both a first input of the instrumentation amplifier 12a and the input of the motion artifact extraction module 9, and the output 3, 4 of the motion artifact extraction module 9 is connected to a second input of the instrumentation amplifier 12a. The output 3, 4 of the motion artifact extraction module 9 comprises a motion artifact signal. The output 7, 8 of the instrumentation amplifier 12a comprises the biopotential electrical signal with the motion artifact signal being reduced. The instrumentation amplifier 12a may be implemented as two differential amplifiers. According to an example embodiment, the instrumentation amplifier 12a may comprise gain adjustment to the output 3, 4 of the motion artifact extraction module 9.

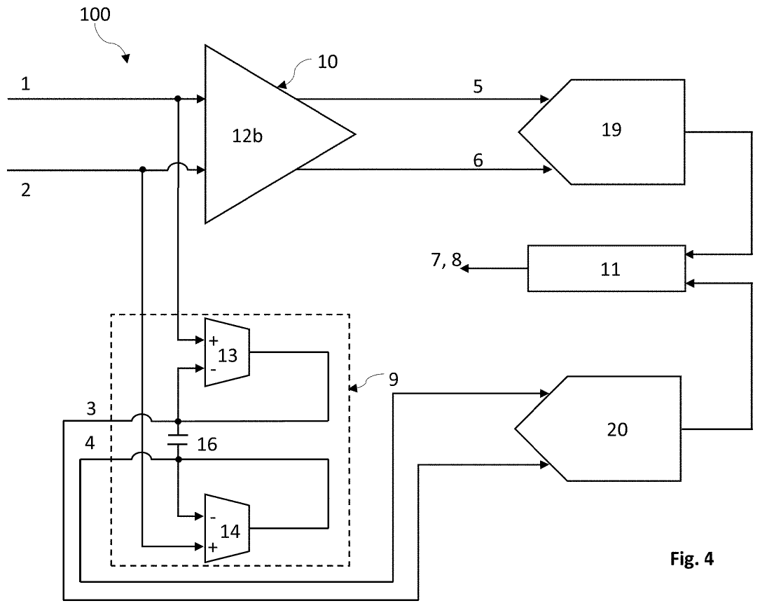

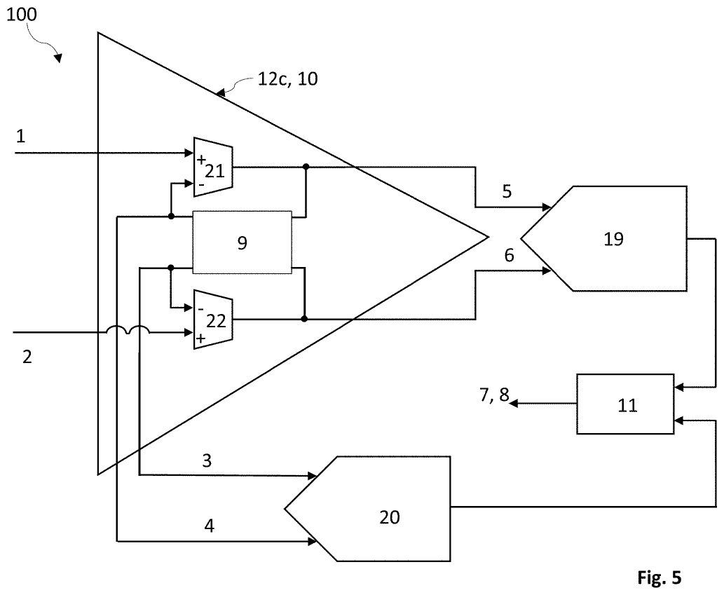

FIG. 4 shows a second schematic diagram of a biosignal monitoring system 100 for reducing a motion artifact from at least one biopotential electrical signal input 1, 2, according to an example embodiment. The system 100 comprises a biopotential electrical signal input 1, 2, an instrumentation amplifier 12b, a motion artifact module 9 including a filter network connected to a first ADC 20, a signal processing module 10 connected to a second ADC (Analog-to-Digital Converter) 19, and a subtraction module 11.

According to an example embodiment, the subtraction module 11 is implemented as a digital domain electronic module.

According to an example embodiment, the signal processing module 10 is implemented as an instrumentation amplifier 12b. The biopotential electrical signal input 1, 2 is connected to the motion artifact extraction module 9. The output 3, 4 of the motion artifact extraction module 9 is connected to the input of the first ADC 20. The output of the first ADC 20 is connected to a first input of the subtraction module 11. The biopotential electrical signal input 1, 2 is also connected to the instrumentation amplifier 12b. The output of the instrumentation amplifier 12b, which is the output 5, 6 of the signal processing module 10, is connected to the input of the second ADC 19. The output of the second ADC 19 is connected to a second input of the subtraction module 11. The output 5, 6 of the signal processing module 10 comprises a digitized differential mode signal and a digitized motion artifact signal. The output 3, 4 of the motion artifact extraction module 9 comprises a digitalized motion artifact signal. The output 3, 4 of the motion artifact extraction module 9 will be subtracted from the output 5, 6 of the signal processing module 10 in the subtraction module 11. The output 7, 8 of the subtraction module 11 comprises the biopotential electrical signal with the motion artifact signal being reduced.

FIG. 5 shows a third schematic diagram of a biosignal monitoring system 100 for reducing a motion artifact from at least one biopotential electrical signal input 1, 2, according to an example embodiment. The system 100 comprises a biopotential electrical signal input 1, 2, an instrumentation amplifier 12c, a third forward path amplifier 21, a fourth forward path amplifier 22, a first ADC 20 and a second ADC 19, a motion artifact extraction module 9, a signal processing module 10, and a subtraction module 11. The signal processing module 10 is implemented as an instrumentation amplifier 12c. The motion artifact extraction module 9 is integrated in the instrumentation amplifier 12c. The subtraction module 11 is implemented as a digital domain electronic module.

According to an example embodiment, the subtraction module 11 is implemented using a least mean squares algorithm. According to an example embodiment, the subtraction module 11 may comprise a gain adjustment function.

According to an example embodiment, the biopotential electrical signal input 1, 2 is connected to the input of the instrumentation amplifier 12c which is the input of the signal processing module 10. The biopotential electrical signal input terminal 1 is connected to the third forward path amplifier 21 and the output of the third forward path amplifier 21 is connected to one input of the motion artifact extraction module 9. The biopotential electrical signal input terminal 2 is connected to the fourth forward path amplifier 22 and the output of the fourth forward path amplifier 22 is connected to the other input of the motion artifact extraction module 9. The output of the forward path amplifiers 21, 22 comprises, in part, the biopotential electrical signal input 1, 2. The output of 3, 4 of the motion artifact extraction module 9 is further connected to the first ADC 20. The output of the first ADC 20 comprises a digitized motion artifact signal. The output of the instrumentation amplifier 12c, which is the output 5, 6 of the signal processing module 10, is further connected to the second ADC 19. The output of the second ADC 19 comprises a digitized differential mode signal and a digitized motion artifact signal. The output of the first ADC 20 is connected to a first input of the subtraction module 11 and the output of the second ADC 19 is connected to a second input of the subtraction module 11. The output 7, 8 of the subtraction module 11 comprises a digitized biopotential electrical signal with the motion artifact signal being reduced.

According to an example embodiment, the input of the motion artifact module 9 may be between any intermediate nodes in the instrumentation amplifier 12c where the input of the motion artifact module 9 comprises a biopotential electrical signal having at least a differential mode signal and a motion artifact signal.

According to an example embodiment, the two forward path amplifiers 21, 22 can be operational amplifiers with closed loop gain control, or they may be instrumentation amplifiers.

According to an example embodiment, the subtraction module 11 may comprise a gain adjustment function. The representation of the motion artifact signal extracted from the motion artifact extraction module 9 may be an attenuated motion artifact signal and the subtraction module 11 may be configured to adjust the gain of the motion artifact signal before it is subtracted from the output 5, 6 of the signal processing module 10.

According to an example embodiment, the subtraction module 11 is implemented using a least mean squares (LMS) algorithm.

According to an example embodiment, a method for reducing a motion artifact from at least one biopotential electrical signal input 1, 2 in the biosignal monitoring system comprises steps of:

extracting a representation of the motion artifact signal from the biopotential electrical signal input 1, 2 by attenuating a differential mode signal present in the biopotential electrical signal input 1, 2 from a first frequency F1 and passing a representation of the motion artifact signal from the biopotential electrical signal input 1, 2 up to a second frequency F2; and subtracting the extracted representation of the motion artifact signal from the biopotential electrical signal input 1, 2.

While some embodiments have been illustrated and described in detail in the appended drawings and the foregoing description, such illustration and description are to be considered illustrative and not restrictive. Other variations to the disclosed embodiments can be understood and effected in practicing the claims, from a study of the drawings, the disclosure, and the appended claims. The mere fact that certain measures or features are recited in mutually different dependent claims does not indicate that a combination of these measures or features cannot be used. Any reference signs in the claims should not be construed as limiting the scope.

What is claimed is:

1. A biosignal monitoring system comprising:
a signal processing module configured to generate a processed biopotential signal based on a biopotential signal input;
a motion artifact extraction module configured to extract a motion artifact signal from the biopotential signal input; and
a subtraction module configured to generate an output based on comparing the motion artifact signal to the processed biopotential signal.

2. The biosignal monitoring system of claim 1, wherein the motion artifact extraction module is implemented as an analog domain electronic circuit.

3. The biosignal monitoring system of claim 1, wherein the motion artifact extraction module comprises a filter network configured for attenuating components of a differential mode signal of the biopotential signal input that are greater than a first frequency and configured for passing single ended components of the biopotential signal input up to a second frequency that is greater than the first frequency.

4. The biosignal monitoring system according to claim 1, wherein the motion artifact extraction module comprises:
a first forward path amplifier having a first input connected to a first biopotential electrical signal input terminal, and a second input and an output connected to a first output terminal of the motion artifact extraction module;
a second forward path amplifier having a first input connected to a second biopotential electrical signal input terminal, a second input and an output connected to a second output terminal of the motion artifact extraction module; and
a capacitor connecting the output of the first forward path amplifier and the output of the second forward path amplifier.

5. The biosignal monitoring system according to claim 4, wherein the capacitor has a capacitance between 100 nF to 100 μF.

6. The biosignal monitoring system according to claim 4, wherein a transconductance of the first forward path amplifier has a value between 10 nS to 1000 nS.

7. The biosignal monitoring system according to claim 4, wherein the first forward path amplifier is implemented as a transconductance amplifier.

8. The biosignal monitoring system according to claim 1, wherein the motion artifact extraction module, the signal processing module, and the subtraction module are implemented as analog domain electronic modules.

9. The biosignal monitoring system according to claim 1, wherein the signal processing module or the subtraction module are implemented as an instrumentation amplifier.

10. The biosignal monitoring system according to claim 9, wherein the biopotential signal input is connected to both a first input of the instrumentation amplifier and an input of the motion artifact extraction module.

11. The biosignal monitoring system according to claim 10, wherein an output of the motion artifact extraction module is connected to a second input of the instrumentation amplifier.

12. The biosignal monitoring system according to claim 1, wherein the subtraction module is implemented as a digital domain electronic module.

13. The biosignal monitoring system according to claim 1, wherein the signal processing module is implemented as an instrumentation amplifier, the biosignal monitoring system further comprising:
a first analog-to-digital converter connected to the output of the motion artifact extraction module and further connected to a first input of the subtraction module; and
a second analog-to-digital connected to the output of the signal processing module and further connected to a second input of the subtraction module.

14. The biosignal monitoring system according to claim 1, wherein the motion artifact extraction module is integrated in an instrumentation amplifier comprising:
a third forward path amplifier connected between a first input terminal of the motion artifact extraction module and the biopotential signal input;
a fourth forward path amplifier connected between a second input terminal of the motion artifact extraction module and the biopotential signal input;
a first analog-to-digital converter connected to the output of the motion artifact extraction module and further connected to a first input of the subtraction module; and
a second analog-to-digital converter connected to the output of the signal processing module and further connected to a second input of the subtraction module.

15. The biosignal monitoring system according to claim 1, wherein the subtraction module is implemented using a least mean squares algorithm.

16. The biosignal monitoring system according to claim 1, wherein the biopotential signal input is an electrocardiogram, electroencephalogram, or electromyogram signal input.

17. A microchip comprising the biosignal monitoring system according to claim 1.

18. A biomedical device comprising the microchip according to claim 17.

19. A method comprising:
extracting a motion artifact signal from a biopotential signal input by attenuating a differential mode signal present in the biopotential signal input from a first frequency and passing the motion artifact signal from the biopotential signal input up to a second frequency; and
comparing the motion artifact signal to the biopotential signal input.

20. The method of claim 19, wherein comparing the motion artifact signal to the biopotential signal input comprises subtracting the motion artifact signal from the biopotential signal input or subtracting the biopotential signal input from the motion artifact signal.
